(19) United States
(12) Patent Application Publication
(10) Pub. No.: US 2008/0081928 A1
(43) Pub. Date: Apr. 3, 2008

(54) Novel Process 470

(75) Inventors: David Ennis, Loughborough (GB); Agnes Ford, Loughborough (GB); Joel LeBars, Loughborough (GB); John Pavey, Loughborough (GB)

Correspondence Address: MORGAN LEWIS & BOCKIUS LLP, 1111 PENNSYLVANIA AVENUE NW, WASHINGTON, DC, 20004, US

(73) Assignee: AstraZeneca AB, Sodertalje (SE)

(21) Appl. No.: 11/850,369

(22) Filed: Sep. 5, 2007

Related U.S. Application Data
(60) Provisional application No. 60/842,447, filed on Sep. 5, 2006.

(51) Int. Cl. C07C 233/57

(52) U.S. Cl. 564/164

(57) ABSTRACT

A process for preparing 2-chloro-5-(3-oxopropyl)-N-(tricyclo[3.3.1.1^{3,7}]dec-1-ylmethyl)-benzamide and compounds of formula (I).

The present invention relates to processes for preparing pharmacologically active compounds and intermediates of use in the preparation of pharmacologically active compounds.

Antagonists of the P2X₇ receptor are of interest for use in the treatment of inflammatory, immune and cardiovascular diseases. International patent application WO 01/44170 describes a series of P2X₇ receptor antagonists and processes for their preparation. One of the processes described in WO 01/44170 employs 2-chloro-5-(3-oxopropyl)-N-(tricyclo[3.3.1.1^{3,7}]dec-1-ylmethyl)-benzamide as an intermediate compound, which is itself prepared through the reaction of 2-chloro-5-iodo-N-(tricyclo[3.3.1.1^{3,7}]dec-1-ylmethyl)-benzamide and allyl alcohol in a palladium catalysed Heck reaction in the presence of a sodium hydrogencarbonate base.

The Heck reaction is the palladium-catalysed arylation of allylic alcohols with aryl halides. Heck reactions often require high temperatures of 100° C. or more, which may cause the decomposition of thermally unstable substrates or products, or induce side reactions such as base catalysed Aldol condensation reactions of the aldehyde products (Heck, J. Org. Chem. p265, Vol. 41, No. 2, 1976; Chalk, J. Org. Chem. p273, Vol. 41, No. 2, 1976). To counter these problems, milder reaction conditions involving the use of inorganic bases such as sodium hydrogencarbonate have been developed, and the use of an inorganic base is now common practice for Heck reactions with sensitive substrates. (Advanced Organic Chemistry, Carey and Sunberg, Third Edition, Part B, p 418-419; and Jeffery, J. Chem. Soc. Chem. Commun., 1984, p1287). Mild reaction conditions may also be achieved via the use of palladium catalysts with tertiary phosphine-ligands, such as Pd[P(t-Bu)₃]₂. However, these complex catalysts are often expensive, difficult to prepare and/or air sensitive, making them unfavourable for use in large-scale commercial syntheses.

The present invention provides an improved process for preparing 2-chloro-5-(3-oxopropyl)-N-(tricyclo[3.3.1.1^{3,7}]dec-1-ylmethyl)-benzamide and its use in a process for preparing some P2X₇ receptor antagonists.

Accordingly, one aspect of the present invention provides a process of preparing 2-chloro-5-(3-oxopropyl)-N-(tricyclo[3.3.1.1^{3,7}]dec-1-ylmethyl)-benzamide, which process comprises reacting 2-chloro-5-iodo-N-(tricyclo[3.3.1.1^{3,7}]dec-1-ylmethyl)-benzamide with allyl alcohol in the presence of a palladium (II) catalyst and a base, which base is of formula NR²R³R⁴, wherein R², R³ and R⁴ each independently represent a C₁₋₆ alkyl group or a C₃₋₆ cycloalkyl group.

In a further aspect, the invention provides a process of preparing a compound of formula (I), or a pharmaceutically acceptable salt thereof, 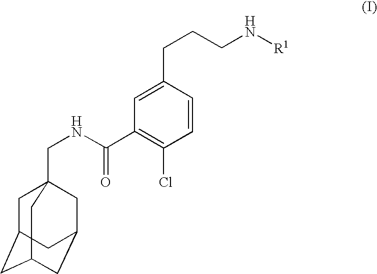
wherein R¹ represents a C₁₋₆ alkyl group which may be optionally substituted by at least one substituent independently selected from hydroxyl and amino; which process comprises:
(a) reacting 2-chloro-5-iodo-N-(tricyclo[3.3.1.1^{3,7}]dec-1-ylmethyl)-benzamide with allyl alcohol in the presence of a palladium (TI) catalyst and a base of formula NR²R³R⁴, wherein R², R³ and R⁴ each independently represent a C₁₋₆ alkyl group or a C₃₋₆ cycloalkyl group, to form 2-chloro-5-(3-oxopropyl)-N-(tricyclo[3.3.1.1^{3,7}]dec-1-ylmethyl)-benzamide;
(b) reacting the 2-chloro-5-(3-oxopropyl)-N-(tricyclo[3.3.1.1^{3,7}]dec-1-ylmethyl)-benzamide so formed with an amine of formula H₂NR¹ and introducing a reducing agent to give a compound of formula (I); and optionally
(c) forming a pharmaceutically acceptable salt of the compound of formula (I).

In the present specification 2-chloro-5-iodo-N-(tricyclo[3.3.1.1^{3,7}]dec-1-ylmethyl)-benzamide may be referred to as compound (A), whilst 2-chloro-5-(3-oxopropyl)-N-(tricyclo[3.3.1.1^{3,7}]dec-1-ylmethyl)-benzamide may be referred to as compound (B), as depicted below:

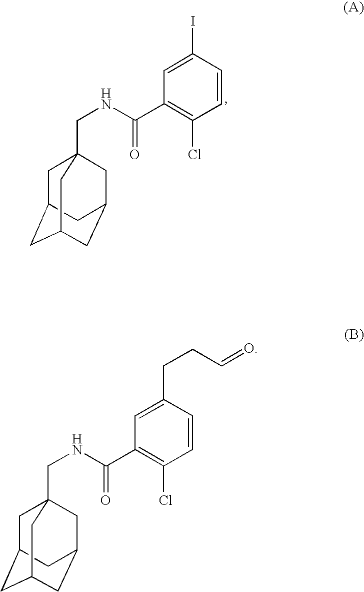

In the compound of formula (I), R¹ represents a C₁₋₆ alkyl group which may be optionally substituted by at least one (e.g. one or two) substituent independently selected from hydroxyl and amino. In an embodiment of the invention R¹ represents a C₁₋₄ alkyl group optionally substituted by one or two hydroxyl groups. Examples of R¹ groups according to this embodiment include CH₂OH, CH₂CH₂OH, CH₂CH₂CH₂OH, CH₂CH₂CH₂CH₂OH, CH₂C(CH₃)₂OH, CH₂CH(OH)CH₃, CH(CH₃)CH₂OH, C(CH₃)(CH₂OH)₂ and C(CH₃)₂CH₂OH.

In the present invention 2-chloro-5-iodo-N-(tricyclo[3.3.1.1^{3,7}]dec-1-ylmethyl)-benzamide (A) is converted to 2-chloro-5-(3-oxopropyl)-N-(tricyclo[3.3.1.1^{3,7}]dec-1-ylmethyl)-benzamide (B) in a reaction using a base of formula NR²R³R⁴, wherein R², R³ and R⁴ each independently represent a C₁₋₆alkyl group or a C₃₋₆cycloalkly group.

For R², R³ and R⁴ examples of C₁₋₆ alkyl groups include linear alkyl groups (e.g. methyl, ethyl, propyl, butyl) and branched alkyl groups (e.g. iso-propyl, tert-butyl); and examples of C₃₋₆ cycloalkyl groups include cyclopropyl, cyclobutyl, cyclopentyl, cyclohexyl.

In an embodiment of the invention, in the base of formula NR²R³R⁴, R² represents a branched C₃₋₄ alkyl group or a C₃₋₆ cycloalkyl group, and R³ and R⁴ each independently represent a C₁₋₆ alkyl group or C₃₋₆ cycloalkyl group. In this embodiment, for R² examples of branched C₃₋₄ alkyl groups include iso-propyl and tert-butyl, and examples of C₃₋₆ cycloalkyl groups include cyclopropyl, cyclobutyl, cyclopentyl and cyclohexyl. For R³ or R⁴ examples of C₁₋₆ alkyl groups include linear alkyl groups (e.g. methyl, ethyl, propyl, butyl), branched alkyl groups (e.g. iso-propyl, tert-butyl) and C₃₋₆ cycloalkyl groups (e.g. cyclopropyl, cyclobutyl, cyclopentyl, cyclohexyl).

Examples of bases of formula NR²R³R⁴ that may be used the present invention include N,N-diisopropylethylamine, N,N-dicyclohexylmethylamine and N,N-diethylcyclohexylamine. In one embodiment of the invention, the base of formula NR²R³R⁴ is N,N-diisopropylethylamine.

Examples of the palladium (TI) catalysts that may be used in the conversion of compound (A) to (B) include palladium (II) acetate and palladium (II) chloride. In one embodiment of the invention, the palladium (II) catalyst is palladium (II) acetate.

In one embodiment of the invention, the palladium (II) catalyst is incorporated directly into the reaction mixture and is not a palladium (II) catalyst generated in situ. In another embodiment of the invention, the palladium (II) catalyst does not comprise a tertiary phosphine-ligand.

An advantageous aspect of the present invention is that the rate of reaction is significantly faster when conducted using a base according to the invention than for comparative processes using inorganic bases. Consequently, by employing the process of the present invention lower quantities of palladium (II) catalyst are required to achieve a given reaction rate than would be required if the reaction was conducted with an inorganic base. Accordingly, in an embodiment of the invention the amount of palladium (II) catalyst present is less than 1 mol % relative to the amount of 2-chloro-5-iodo-N-(tricyclo[3.3.1.1^{3,7}]dec-1-ylmethyl)-benzamide (A). When the palladium catalyst is palladium (II) acetate, the amount of palladium catalyst may be less than 0.5 mol % relative to A.

The conversion of compound (A) into compound (B) according to the present invention may be conducted in any suitable solvent. Examples of suitable solvents include hydrocarbon solvents such as toluene and ethers such as tetrahydrofuran, 2-methyltetrahydrofuran, di-n-butylether, methyl-tert-butyl ether and mixtures thereof. Other solvents that may be used include isopropylacetate, 4-methyl-2-pentanone, tert-butylalcohol, 4-methyl-2-pentanol, diethoxymethane, acetonitrile and mixtures thereof. In one embodiment of the invention the solvent is toluene, tetrahydrofuran or 2-methyltetrahydrofuran. In another embodiment, the solvent is toluene.

In the present invention the conversion of compound (A) into compound (B) may be conducted at any suitable temperature, but is conveniently conducted at temperatures of less than 100° C. For example, in one embodiment of the invention the reaction is conducted at a temperature of from 20 to 95° C. In another embodiment of the invention the reaction is conducted at a temperature of from 50 to 90° C. In a still further embodiment the reaction is conducted at a temperature of from 70 to 85° C.

In an embodiment of the invention the conversion of compound (A) to (B) may be facilitated with the use of a phase transfer catalyst. Examples of phase transfer catalysts that may be used include tetrabutyl ammonium chloride (Bu₄NCl), tetrabutyl ammonium bromide (Bu₄NBr), tetrabutyl ammonium iodide (Bu₄NI), tetrabutyl ammonium sulfate [(Bu₄N)₂SO₄] and tetrabutyl ammonium hydrogensulfate (Bu₄NHSO₄). In an embodiment of the invention the conversion of (A) to (B) is facilitated by use of a phase transfer catalyst selected from tetrabutyl ammonium chloride (Bu₄NCl), tetrabutyl ammonium bromide (Bu₄NBr) or tetrabutyl ammonium iodide (Bu₄NI). In another embodiment of the invention the phase transfer catalyst is tetrabutyl ammonium chloride (Bu₄NCl). When present the molar ratio of phase transfer catalyst to compound (A) may conveniently be in range of 5:1 to 1:5. Good results may be achieved using approximately stoichiometric amounts of phase transfer catalyst and compound (A).

Compound (B) may be isolated using standard techniques known in the art, and subsequently converted into compounds of formula (I), or used in situ to prepare compounds of formula (I).

Compound (B) may react with itself and other aldehyde by-products in the reaction mixture in Aldol type reactions. Products of these Aldol reactions are a common source of impurity in the preparation of (B). The most common of the Aldol impurities are N-(1-adamantylmethyl)-5-[4-(3-{[(1-adamantylmethyl)amino]carbonyl}-4-chlorobenzyl)-3-hydroxy-5-oxopentyl]-2-chlorobenzamide [formed by the based catalysed Aldol reaction of product (B)], and 3,3′-[(2Z)-2-formylpent-2-ene-1,5-diyl]bis[N-(1-adamantylmethyl)-6-chlorobenzamide] [formed by dehydration of the afore mentioned Aldol product]. Other minor Aldol impurities result from the formation of branched aldehyde N-(1-adamantylmethyl)-2-chloro-5-(1-methyl-2-oxoethyl)benzamide and its subsequent reaction with other aldehydes in the reaction mixture. By employing the process of the present invention amounts of Aldol impurities may be reduced compared to alternative processes. Compounds of formula (I) may be prepared by reacting compound (B), formed in accordance with the present invention, with an amine of formula H₂NR¹, and introducing a reducing agent. In this aspect of the invention the reducing agent may conveniently be introduced to the reaction after the amine of formula H₂NR¹ has reacted with compound (B), however, in certain embodiments it may also be introduced into the reaction before, consecutively or immediately after the addition of the amine of formula H₂NR¹.

In the present invention the reaction of compound (B) with an amine of formula H₂NR¹ may be conducted in any suitable solvent. Examples of suitable solvents include toluene, and isopropanol or mixtures thereof.

The reaction of compound (B) with an amine of formula H₂NR¹ may be conducted at any suitable temperature. For example, in one embodiment of the invention the reaction is conducted at a temperature of from 0 to 100° C. In another embodiment of the invention the reaction is conducted at a temperature of from 20 to 70° C.

Examples of reducing agents that may be used according to the present invention include sodium triacetoxyborohydride [NaBH(OAc)₃], sodium borohydride/acetic acid [NaBH₄/AcOH], and hydrogen. When hydrogen is the reducing agent it will normally be used in the presence of a suitable catalyst (e.g. a palladium, platinum, iridium or nickel catalyst). In an embodiment of the invention the reducing agent is hydrogen in the presence of platinum on a carbon support [Pt/C]). Hydrogen and Pt/C may be conveniently introduced after compound (B) has reacted with the amine of formula (I), and the reduction may be conveniently conducted at a temperature in the range of from 20 to 80° C., and at a hydrogen pressure of 1 to 5 BarG (200 to 500 kPaG).

Compounds of formula (I) may be isolated, or optionally converted into pharmaceutically acceptable salts thereof, using standard techniques known in the art. Examples of pharmaceutically acceptable salts include acid addition salts derived from pharmaceutically acceptable inorganic and organic acids such as a chloride, bromide, sulphate, phosphate, maleate, fumarate, tartrate, citrate, benzoate, 4-methoxybenzoate, 2- or 4-hydroxybenzoate, 4-chlorobenzoate, p-toluenesulphonate, methanesulphonate, ascorbate, acetate, succinate, lactate, glutarate, gluconate, tricarballylate, hydroxynaphthalene-carboxylate or oleate salt.

It will be appreciated by those skilled in the art that in the processes of the present invention certain functional groups such as hydroxyl, or amino groups in the starting reagents or intermediate compounds may need to be protected by protecting groups. Thus, the processes may involve at certain stages the introduction and/or removal of one or more protecting groups. The protection and deprotection of functional groups is described in ‘Protective Groups in Organic Synthesis’, 2nd edition, T. W. Greene and P. G. M. Wuts, Wiley-Interscience (1991) and ‘Protecting Groups’, P. J. Kocienski, Georg Thieme Verlag (1994). It will further be appreciated by those skilled in the art that in certain embodiments of the invention, in order to optimise the processes, additional purification steps and/or further reaction components may be employed.

2-Chloro-5-iodo-N-(tricyclo[3.3.1.1^{3,7}]dec-1-ylmethyl)-benzamide (A) may be prepared by known chemistry, for example from 2-chloro-5-iodobenzoic acid and 1-adamantanemethylamine in chemistry according or analogous to that described in WO01/44170.

In the present invention particularly good results may be achieved when the amount of residual 1-adamantanemethylamine present in compound (A) is kept to a minimum. Therefore, in one embodiment of the present invention the amount of 1-adamantanemethylamine present in compound (A) is less than 1% wt. In another embodiment the amount of 1-adamantanemethylamine present in compound (A) is less than 0.1% wt.

The invention will now be further explained by reference to the following illustrative examples.

Preparation 2-Chloro-5-iodo-N-(tricyclo[3.3.1.1^{3,7}]dec-1-ylmethyl)-benzamide (Compound A)

5-Iodo-2-chlorobenzoic acid (40.00 g, 141.6 mmol) was charged to a 500 ml reaction vessel, followed by Bu₄NCl (0.40 g, 0.01 eq, 1.42 mmol) and toluene (80 ml, 2 vol) under an inert atmosphere (N₂). The suspension was heated to 70-75° C., then thionyl chloride (12.40 ml, 1.2 eq, 169.94 mmol) was added drop-wise over 30-60 min. The resulting suspension is heated at 70-75° C. for approximately 3 hours. The reaction was monitored by HPLC (MeOH quench of sample) and on completion the reaction mixture, now a clear solution of 5-iodo-2-chlorobenzoyl chloride, was cooled to 20-25° C.

1-Adamantanemethylamine.HCl (28.56 g, 1.0 eq, 141.62 mmol), toluene (40 ml, 1.0 vol) and 5M aqueous NaOH (84.96 ml, 3.0 eq, 424.82 mmol) were charged to a second vessel (1000 ml) and heated to 75-80° C. under an inert atmosphere (N₂). The solution of 5-iodo-2-chlorobenzoyl chloride, was added drop-wise maintaining the temperature at 75-80° C. The residues were washed in with toluene (10 ml, 0.25 vol). The reaction was monitored by HPLC and on completion water (40 ml, 1 vol) was added and the aqueous phase separated. A second charge of water (40 ml, 1 vol) was added, the mixture cooled to 60-65° C. and then n-heptane (240 ml, 6 vol) added. The suspension obtained was stirred at 60-65° C. then cooled to 20-25° C. and stirred for an additional 2 hours. The suspension was filtered and the cake washed with water (80 ml×2, 2 vol×2) followed by n-heptane (80 ml, 2 vol). The white to off-white solid obtained was dried in an oven at 40-45° C. under vacuum. Yield of 2-Chloro-5-iodo-N-(tricyclo [3.3.1.1^{3,7}] dec-1-ylmethyl)-benzamide (Compound A) was 58.42 g. The amount of residual amine starting material remaining was 0.026% w/w (determined by gas chromatography).

Preparation of 2-Chloro-5-(3-oxopropyl)-N-(tricyclo[3.3.1.1^{3,7}]dec-1-ylmethyl)-benzamide (Compound B)

The reaction of 2-chloro-5-iodo-N-(tricyclo[3.3.1.1^{3,7}]dec-1-ylmethyl)-benzamide (compound A) and allyl alcohol in the presence of a palladium (II) catalyst was conducted using a variety of different bases as listed in Table 1. General reaction conditions were as follows: 2-chloro-5-iodo-N-(tricyclo[3.3.1.1^{3,7}]dec-1-ylmethyl)-benzamide, Bu₄NCl (1.05 eq), Pd(OAc)₂, toluene and base were charged to a flask under an inert atmosphere (N₂ or Ar), followed by allyl alcohol (1.25 eq). The reaction was heated at 80-85° C. The reaction was sampled after 1 hour and after overnight reaction, and HPLC used to monitor consumption of 2-chloro-5-iodo-N-(tricyclo[3.3.1.1^{3,7}]dec-1-ylmethyl)-benzamide, formation of 2-chloro-5-(3-oxopropyl)-N-(tricyclo [3.3.1.1^{3,7}]dec-1-ylmethyl)-benzamide and formation of impurities. Quantities of reagent quoted in equivalents are mol eq to compound (A).

HPLC conditions were as follows: Column: Genesis C18, 10 cm×3 mm, 3 μm. Mobile Phase A: 0.1% TFA aq. Mobile Phase B:0.1% TFA aq in 90% MeCN. Flow rate: 0.6 ml/min. Oven temperature:45° C. Wavelength: 225 nm, 4 nm bandwidth; reference wavelength 380 nm, 100 nm bandwidth. Injection volume: 2.5 μl. Run time: 21 min. Equilibration Time: 5 min Gradient: Time (min)/% B; 0 min/10.0; 1 min/10.0; 11 min/90.0; 21 min/90.0. Mobile phase A: 0.1% TFA aq (1 ml of TFA diluted in 1 litre); degas if necessary. Mobile phase B: Mix 1 ml of TFA, 100 ml of purified water and 900 ml of HPLC grade acetonitrile; degas if necessary. Sample preparation: each sample is made of a few drops of the reaction mixture diluted in 1 ml of methanol. The results are shown in Table 1.

From Table 1 it can be seen that when the reaction was conducted with tertiary amine bases (according to the invention) high conversions to product (B) were obtained using a 10 fold lower amount of palladium catalyst than that required for the reaction with NaHCO₃ (0.2 mol % from 2 mol %). Moreover, when conducted according to the invention the reaction mixture was more stable after overnight reaction than when the reaction was conducted with the secondary amine base Cy₂NH, the use of which resulted in large amounts of Aldol impurity being formed.

TABLE 1

| | eq of | After 1 hour | | Overnight | | Pd(OAc)₂ |
|---|---|---|---|---|---|---|
| Base | Base | % (B) | % Imp | % (B) | % Imp | mol % |
| NaHCO₃ | 2.5 | 82 * | <1 | 78 | 3 | 2 |
| Cy₂NH | 2.5 | 80 * | 11 | 36 | 42 | 0.2 |
| iPr₂EtN | 1.5 | 88 * | <1 | 88 | 5 | 0.2 |
| Cy₂McN | 1.5 | 86 * | 2 | 76 | 13 | 0.2 |
| Et₂CyN | 1.5 | 87 | 1 | 78 | 13 | 0.2 |
| Et₃N | 1.2 | 83 * | <1 | 76 | 7 | 0.2 |
| Bu₃N | 1.5 | 67 | <1 | 83 | 8 | 0.2 |

* Complete reaction: no starting material detected by HPLC
Cy: Cy is cyclohexyl

In Table 1 ‘% B’ denotes the amount of 2-chloro-5-(3-oxopropyl)-N-(tricyclo[3.3.1.1^{3,7}]dec-1-ylmethyl)-benzamide product as a percentage of total product and total reaction impurity as determined by HPLC. ‘% Imp’ denotes the amount of Aldol impurities as a percentage of total product and total reaction impurity, the major Aldol impurities being N-(1-adamantylmethyl)-5-[4-(3-{[(1-adamantylmethyl)amino]carbonyl}-4-chlorobenzyl)-3-hydroxy-5-oxopentyl]-2-chlorobenzamide and 3,3′-[(2Z)-2-formylpent-2-ene-1,5-diyl]bis[N-(1-adamantylmethyl)-6-chlorobenzamide]. Amounts of other (non-Aldol) impurities detected, predominately the branched aldehyde N-(1-Adamantylmethyl)-2-chloro-5-(1-methyl-2-oxoethyl)benzamide, did not vary significantly under the various reaction conditions.

1: A process of preparing 2-chloro-5-(3-oxopropyl)-N-(tricyclo[3.3.1.1^{3,7}]dec-1-ylmethyl)-benzamide, which process comprises reacting 2-chloro-5-iodo-N-(tricyclo[3.3.1.1^{3,7}]dec-1-ylmethyl)-benzamide with allyl alcohol in the presence of a palladium (II) catalyst and a base, which base is of formula NR²R³R⁴, wherein R², R³ and R⁴ each independently represent a C₁₋₆ alkyl group or a C₃₋₆ cycloalkyl group.

2: The process according to claim 1, wherein the base is of formula NR²R³R⁴, wherein R² represents a branched C₃₋₄ alkyl group or a C₃₋₆ cycloalkyl group and R³ and R⁴ each independently represent a C₁₋₆ alkyl group or C₃₋₆ cycloalkyl group.

3: The process according to claim 2, wherein the base of formula NR²R³R⁴ is selected from N,N-diisopropylethylamine, N,N-dicyclohexylmethylamine or N,N-diethylcyclohexylamine.

4: The process according to claim 1 wherein the palladium (II) catalyst is palladium (II) acetate.

5: The process according to claim 1, wherein the amount of palladium (II) catalyst present is less than 1 mol % relative to the amount of 2-chloro-5-iodo-N-(tricyclo[3.3.1.1^{3,7}]dec-1-ylmethyl)-benzamide.

6: The process according to claim 1, wherein the reaction of 2-chloro-5-iodo-N-(tricyclo[3.3.1.1^{3,7}]dec-1-ylmethyl)-benzamide with allyl alcohol is conducted at a temperature of less than 100° C.

7: A process of preparing a compound of formula (I), or a pharmaceutically acceptable salt thereof, 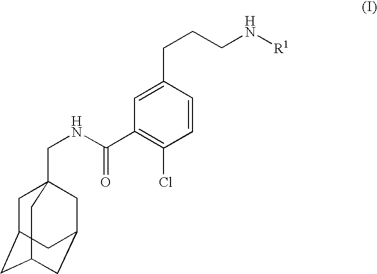
wherein R¹ represents a C₁₋₆ alkyl group which may be optionally substituted by at least one substituent independently selected from hydroxyl and amino;
which process comprises:
(a) reacting 2-chloro-5-iodo-N-(tricyclo[3.3.1.1^{3,7}]dec-1-ylmethyl)-benzamide with allyl alcohol in the presence of a palladium (II) catalyst and a base of formula NR²R³R⁴, wherein R², R³ and R⁴ each independently represent a C₁₋₆ alkyl group or a C₃₋₆ cycloalkyl group, to form 2-chloro-5-(3-oxopropyl)-IV-(tricyclo[3.3.1.1^{3,7}]dec-1-ylmethyl)-benzamide;
(b) reacting the 2-chloro-5-(3-oxopropyl)-N-(tricyclo[3.3.1.1^{3,7}]dec-1-ylmethyl)-benzamide so formed with an amine of formula H₂NR¹ and introducing a reducing agent to give a compound of formula (I); and optionally
(c) forming a pharmaceutically acceptable salt of the compound of formula (I).

8: The process according to claim 7, wherein R¹ represents a C₁₋₄ alkyl group optionally substituted by one or two hydroxyl groups.

9: The process according to claim 8, wherein R¹ represents CH₂OH, CH₂CH₂OH, CH₂CH₂CH₂OH, CH₂CH₂CH₂CH₂OH, CH₂C(CH₃)₂OH, CH₂CH(OH)CH₃, CH(CH₃)CH₂OH, C(CH₃)(CH₂OH)₂ or C(CH₃)₂CH₂OH.

10: The process according to claim 7, wherein the base is of formula NR²R³R⁴, wherein R² represents a branched C₃₋₄ alkyl group or a C₃₋₆ cycloalkyl group and R³ and R⁴ each independently represent a C₁₋₆ alkyl group or C₃₋₆ cycloalkyl group.

11. The process according to claim 7, wherein the base of formula NR²R³R⁴ is selected from N,N-diisopropylethylamine, N,N-dicyclohexylmethylamine or N,N-diethylcyclohexylamine.

12: The process according to claim 7, wherein the palladium (II) catalyst is palladium (II) acetate.

13: The process according to claim 7, wherein the amount of palladium (II) catalyst present is less than 1 mol % relative to the amount of 2-chloro-5-iodo-N-(tricyclo[3.3.1.1^{3,7}]dec-1-ylmethyl)-benzamide.

14: The process according to claim 7, wherein the reaction of 2-chloro-5-iodo-N-(tricyclo[3.3.1.1^{3,7}]dec-1-ylmethyl)-benzamide with allyl alcohol is conducted at a temperature of less than 100° C.
